(19) United States
(12) Patent Application Publication
(10) Pub. No.: US 2007/0009428 A1
(43) Pub. Date: Jan. 11, 2007

(54) Compounds and methods for enhanced delivery to disease targets

(76) Inventors: Faisal Ahmed Syud, Clifton Park, NY (US); Nichole Lea Wood, Niskayuna, NY (US); Andrew Soliz Torres, Clifton Park, NY (US); Mark Thomas Baillie, Atlanta, GA (US); Dinko Eduardo Gonzalez Trotter, Clifton Park, NY (US); Cristina Tan Hehir, Niskayuna, NY (US); Ming Zhao, Clifton Park, NY (US); Daniel Joshua Kramer, Ballston Lake, NY (US)

Correspondence Address: GENERAL ELECTRIC COMPANY;GLOBAL RESEARCH, PATENT DOCKET RM. BLDG. K1-4A59, NISKAYUNA, NY, 12309, US

(21) Appl. No.: 11/175,668

(22) Filed: Jul. 6, 2005

(51) Int. Cl. A61K 51/00

(52) U.S. Cl. 424/1.11; 424/1.69

(57) ABSTRACT

A set of compounds that includes an active-agent labeled species and a pretargeting conjugate is disclosed. The active agent-labeled species includes a ligand coupled to an active agent. The pretargeting conjugate includes a protein conjugated to a targeting species having a targeting moiety capable of binding to an in-vivo target or a biomarker produced by or associated with the target. The protein is substantially free of a cofactor. Also disclosed are methods of administering the pretargeting species and the active-agent labeled species to a subject for diagnosing or treating a disease condition, or assessing the effectiveness of a treatment of the disease condition.

### BACKGROUND OF THE INVENTION

The invention relates to compounds for enhanced delivery to disease targets. In particular, the invention relates to such compounds for enhanced delivery of diagnostic or therapeutic agents to disease sites based on a pretargeting strategy.

The growing need for the early diagnosis and assessment and/or treatment of disease can potentially be addressed by pharmaceuticals that preferentially accumulate at the disease sites. In diagnostic applications, these pharmaceuticals can elucidate the state of the disease through its distinctive molecular biology expressed as disease markers that are not present, or are present in diminished levels, in healthy tissues. In therapeutic applications, these pharmaceuticals can deliver an enhanced dose of therapeutic agents to the disease sites through specific interactions with the disease markers. By specifically targeting physiological or cellular functions that are present only in disease states, these pharmaceuticals can report exclusively on the scope and progress of that disease or exclusively target the diseased tissue. A signal-generating moiety is a key element of these diagnostic pharmaceuticals, which produce differentiated signals at the disease sites.

The detection of a target site benefits from a high signal-to-background ratio of detection agent. Therapy benefits from as high an absolute accretion of therapeutic agent at the target site as possible, as well as a reasonably long duration of binding. In order to improve the targeting ratio and amount of agent delivered to a target site, the use of targeting vectors comprising diagnostic or therapeutic agents conjugated to a targeting moiety for preferential localization is known.

Examples of targeting vectors include diagnostic or therapeutic agent conjugates of targeting moieties such as antibody or antibody fragments, cell-or tissue-specific peptides, and hormones and other receptor-binding molecules. For example, antibodies against different determinants associated with pathological and normal cells, as well as associated with pathogenic microorganisms, have been used for the detection and treatment of a wide variety of pathological conditions or lesions. In these methods, the targeting antibody is directly conjugated to an appropriate detecting or therapeutic agent.

One problem encountered in direct targeting methods, i.e., in methods wherein the active agent, such as a diagnostic or therapeutic active agent, is conjugated directly to the targeting moiety and administered simultaneously, is that a relatively small fraction of the conjugate actually binds to the target site, while the majority of conjugate remains in circulation and compromises in one way or another the function of the targeted conjugate (i.e., the conjugate accumulated or bound at the target). In the case of a diagnostic conjugate (e.g., a radioimmunoscintigraphic or magnetic resonance imaging conjugate), the non-targeted conjugate, which remains in circulation, can increase background and decrease resolution. In the case of a therapeutic conjugate having a toxic therapeutic agent (e.g., a radioisotope, drug, or toxin) attached to a long-circulating targeting moiety such as an antibody, circulating conjugate can result in toxicity to the host, such as marrow toxicity or systemic side effects.

Pretargeting methods have been developed to increase the target-to-background ratios and improve resolution. In pretargeting methods, a primary targeting species (which is not bound to an active agent) is targeted to an in-vivo target site. The primary targeting species comprises a first targeting moiety, which binds to the target site, and a second moiety, which presents a binding site available for binding by a subsequently administered second targeting species. Once sufficient accretion of the primary targeting species is achieved, the second targeting species comprising a diagnostic or therapeutic active agent and a second targeting moiety, which recognizes the available binding site of the primary targeting species, is administered.

Pretargeting strategy offers certain advantages over the use of direct targeting methods. For example, use of the pretargeting strategy for the in-vivo delivery of radionuclides to a target for therapy, e.g., radioimmunotherapy, reduces the marrow toxicity caused by prolonged circulation of a radioimrnmunoconjugate. This is because the radioisotope is delivered as a rapidly clearing, low molecular weight chelate rather than directly conjugated to a primary targeting molecule, which is often a long-circulating species.

Despite these advantages, known pretargeting strategies still suffer from certain drawbacks. One disadvantage is the very low amount of active agent delivered to the target site compared to when the active agent is directly attached to an antibody, for a variety of reasons. Another disadvantage is that the active agent-carrying vectors, which are often peptides, are often degraded by endogenous proteases in the body. Furthermore, when conjugated to antibodies, the active agent can generate antibodies in a patient.

### SUMMARY OF THE INVENTION

The purpose and advantages of embodiments of the invention will be set forth and apparent from the description that follows, as well as will be learned by practice of the embodiments of the invention. Additional advantages will be realized and attained by the methods and systems particularly pointed out in the written description and claims hereof, as well as from the appended drawings.

Diagnostic compounds designed for use in a pretargeting strategy comprising a ligand and an enzyme are disclosed.

Accordingly, one aspect of the invention includes a set of compounds comprising an active agent-labeled species and a pretargeting conjugate. The active agent-labeled species includes a ligand coupled with an active agent selected from a group consisting of diagnostic active agents, therapeutic active agents, and combinations thereof. The pretargeting conjugate includes a protein that is conjugated to a targeting species having a targeting moiety capable of binding to an in-vivo target or a biomarker substance produced by or associated with the target. The protein is substantially free of a cofactor.

A second aspect of the invention includes a method for diagnosing or treating a disease condition. The method includes (i) administering a pretargeting conjugate to a subject, (ii) allowing the pretargeting conjugate to localize at a target; and (iii) administering an active agent-labeled species to the subject. The pretargeting conjugate includes a protein conjugated to a targeting species having a targeting moiety that binds to an in-vivo target or a biomarker substance produced by or associated with the target. The protein is substantially free of a cofactor. The active agent-labeled species includes a ligand coupled with an active agent selected from a group consisting of diagnostic active agents, therapeutic active agents, and combinations thereof. The active agent is capable of performing a function selected from elucidating the disease condition and reducing an adverse effect of the disease condition.

A third aspect of the invention includes a method for diagnosing or treating a disease condition. The method includes (i) obtaining a base-line image of a portion of a subject suspected of having the disease condition; (ii) administering a pretargeting conjugate to the subject; (iii) allowing the pretargeting conjugate to localize at the target; (iv) administering an active agent-labeled species to the subject; (v) obtaining an additional image of the same portion of the subject; and (vi) comparing the base-line image with the additional image to evaluate the disease condition. The pretargeting conjugate includes a protein conjugated to a targeting species having a targeting moiety that binds to an in-vivo target or a biomarker substance produced by or associated with the target. The protein is substantially free of a cofactor. The active agent-labeled species includes a ligand coupled with an active agent selected from a group consisting of diagnostic active agents, therapeutic active agents, and combinations thereof. The active agent is capable of performing a function selected from a group consisting of elucidating the disease condition and reducing an adverse effect of the disease condition.

A fourth aspect of the invention includes a method for assessing an effectiveness of a prescribed regimen for treating a disease condition that is characterized by an overproduction or underproduction of a disease-specific substance or biomarker. The method includes: (i) obtaining a base-line image of a portion of a subject suspected of having the disease condition; (ii) administering a pretargeting conjugate to the subject; (iii) allowing the pretargeting conjugate to localize at the target; and (iv) administering an active agent-labeled species to the subject; (v) obtaining a pre-treatment image coming from the same portion of the subject; (vi) treating the disease condition in the subject with a prescribed regimen; (vii) repeating steps (ii), (iii), and (iv); and (viii) obtaining a post-treatment image coming from the same portion of the subject as in step (v). The pretargeting conjugate includes a protein conjugated to a targeting species having a targeting moiety that binds to an in-vivo target or a biomarker substance produced by or associated with the target. The protein is substantially free of a cofactor. The active agent-labeled species includes a ligand coupled with an active agent selected from a group consisting of diagnostic active agents, therapeutic active agents, and combinations thereof. The active agent is capable of performing a function selected from a group consisting of elucidating the disease condition and reducing an adverse effect of the disease condition.

The accompanying figures, which are incorporated in and constitute part of this specification, are included to illustrate and provide a further understanding of the method and system of the invention. Together with the description, the figures serve to explain the principles of the invention.

### BRIEF DESCRIPTION OF THE DRAWINGS

FIG. 1A is a schematic representation of a pair of active agent-labeled species and pretargeting conjugate in accordance with an embodiment of the invention;

FIG. 1B is another schematic representation of a pair of active agent-labeled species and pretargeting conjugate in accordance with an embodiment of the invention;

FIG. 2 is a schematic representation of a pair of active agent-labeled species and pretargeting conjugate attached to a target in accordance with an embodiment of the invention;

FIG. 3 is a flow chart of a method for diagnosing or treating a disease condition in accordance with an embodiment of the invention; and

FIG. 4 is another flow chart of a method for diagnosing or treating a disease condition in accordance with an embodiment of the invention.

### DETAILED DESCRIPTION OF THE INVENTION

Reference will now be made in detail to exemplary embodiments of the invention, which are illustrated in the accompanying figures and examples. Referring to the drawings in general, it will be understood that the illustrations are for the purpose of describing a particular embodiment of the invention and are not intended to limit the invention thereto.

Whenever a particular embodiment of the invention is said to comprise or consist of at least one element of a group and combinations thereof, it is understood that the embodiment may comprise or consist of any of the elements of the group, either individually or in combination with any of the other elements of that group. Furthermore, when any variable occurs more than one time in any constituent or in formula, its definition on each occurrence is independent of its definition at every other occurrence. Also, combinations of substituents and/or variables are permissible only if such combinations result in stable compounds.

The invention provides diagnostic compounds or pharmaceuticals designed for use in a pretargeting strategy.

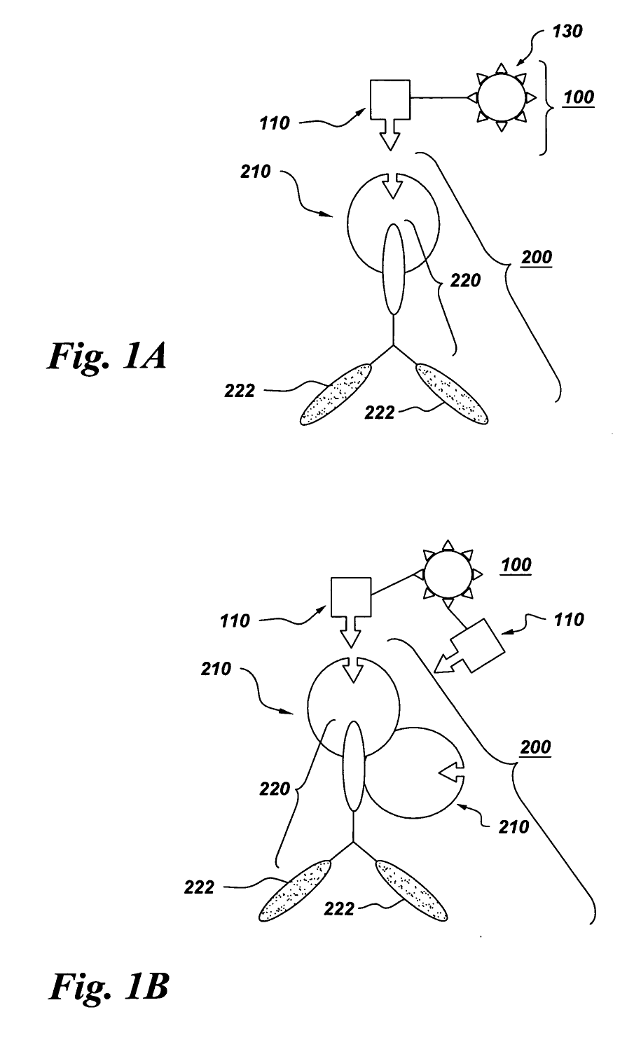

With reference to FIG. 1A, there is shown one embodiment of a set of compounds comprising an active agent-labeled species 100 and a pretargeting conjugate 200. The active agent-labeled species 100 includes a ligand 110 coupled with an active agent 130. The active agent-labeled species 100 may include one or more ligands 100 coupled to active agent 130. The pretargeting conjugate 200 includes a protein 210 that is conjugated to a targeting species 220 having a targeting moiety 222 capable of binding to an in-vivo target or a biomarker produced by or associated with the in-vivo-target. The protein is substantially free of a cofactor. It should be appreciated that the active agent-labeled species 100 can include one or more ligands 110 and one or more diagnostic active agents 130, as shown in FIG. 1B. It should also be appreciated that the pretargeting conjugate 200 can include one or more proteins 210, one or more targeting species 220, wherein a targeting species 220 has one or more targeting moieties 222, as shown in FIG. 1B. When any variable, such as the protein, ligand, or targeting species, occurs more than one time in any constituent or in formula, its definition on each occurrence is independent of its definition at every other occurrence, unless otherwise noted.

Active Agent Labeled Species

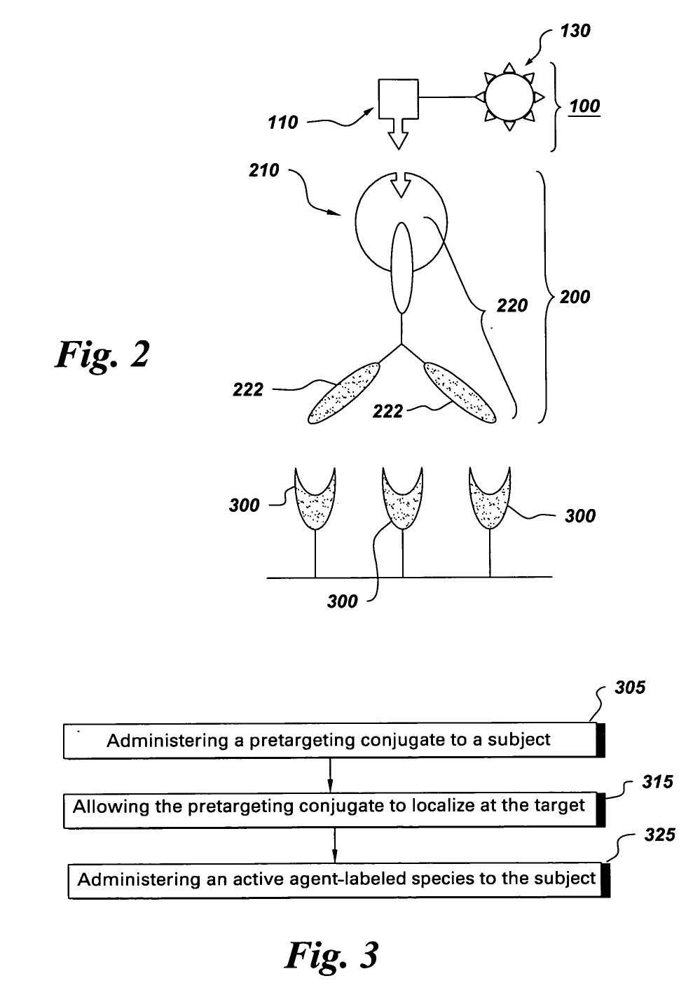

Embodiments of the active agent labeled species are shown in FIG. 1A, FIG. 1B, and FIG. 2 and represented by the schema below:

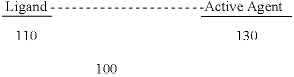

In one embodiment, the ligand includes a ligand such as, but not limited to, a sulfonate ester derivative, an α-chloroacetamide derivative, an α-chloroacetamide derivative on a peptide, a fluorophosphonate or a fluorophosphate derivative, a matrix metalloprotease inhibitor ilomastat, zinc-chelating hydroxamate coupled to benzophenone photocrosslinker, sublactam, difluoromethylphenyl phosphate (DFPP), α-bromobenzylphosphonate, β-lactamase inhibitor, rapamycin, FK506 FK1012, AP1510, AP1903, AP20187, ubiquitin, tyr-phosphate mimic, (5′-p-fluorosulfonylbenzoyl adenosine (FSBA), and a fatty acid synthase inhibitor, either individually or in any combinations thereof. Also included are any derivatives of the ligands, either individually or in any combinations thereof.

In a particular embodiment, the ligand includes, but is not limited to, a sulfonate ester derivative, a α-chloroacetamide derivative, a α-chloroacetamide derivative, and fluorophosphonate, either individually or in combinations thereof. Furthermore, when the active agent labeled species includes more than one ligand, the ligands at each occurrence are independent of the ligands at every other occurrence.

Diagnostic and Therapeutic Active Agents

Among the diagnostic and therapeutic active agents applicable to and useful in the present invention, gamma-emitters, positron-emitters, x-ray emitter, paramagnetic ions and fluorescence-emitters are suitable for detection and/or therapy, while beta- and alpha-emitters and neutron-capturing agents, such as boron and uranium, also can be used for therapy.

Therapeutic Agents

Examples of therapeutic active agents are isotopes, drugs, toxins, fluorescent dyes activated by nonionizing radiation, hormones, hormone antagonists, receptor antagonists, enzymes or proenzymes activated by another agent, autocrine, or cytokine. Many drugs and toxins are known which have cytotoxic effects on cells. They can be found in compendia of drugs and toxins, such as the Merck Index, Goodman and Gilman's “The Pharmacological Basis of Therapeutics” (Tenth Edition, McGraw-Hill, New York, 2001), and the like, and in the references cited in U.S. patents. Any such drug can be conjugated, coupled, attached to, or loaded onto the ligand of the present invention by conventional means and/or chemistry well known in the art. Specific embodiments of such conjugation, coupling, attachment, or loading are disclosed herein below.

Dyes used, for example, in photodynamic therapy, conjugated to ligands used in conjunction with appropriate nonionizing radiation are also contemplated.

The use of light and porphyrins is also contemplated and their use in cancer therapy has been reviewed by van den Bergh (Chemistry in Britain, May 1986, Vol. 22, pp. 430-437.

Examples of cytotoxic agents are listed in Goodman and Gilman's “The Pharmacological Basis of Therapeutics,” Tenth Edition, McGraw-Hill, New York, 2001. These include taxol; nitrogen mustards, such as mechlorethamine, cyclophosphamide, melphalan, uracil mustard and chlorarnbucil; ethylenimine derivatives, such as thiotepa; alkyl sulfonates, such as busulfan; nitrosoureas, such as carmustine, lomustine, semustine and streptozocin; triazenes, such as dacarbazine; folic acid analogs, such as methotrexate; pyrimidine analogs, such as fluorouracil, cytarabine and azaribine; purine analogs, such as mercaptopurine and thioguanine; vinca alkaloids, such as vinblastine and vincristine; antibiotics, such as dactinomycin, daunorubicin, doxorubicin, bleomycin, mithramycin and mitomycin; enzymes, such as L-asparaginase; platinum coordination complexes, such as cisplatin; substituted urea, such as hydroxyurea; methyl hydrazine derivatives, such as procarbazine; adrenocortical suppressants, such as mitotane; hormones and antagonists, such as adrenocortisteroids (prednisone), progestins (hydroxyprogesterone caproate, medroprogesterone acetate and megestrol acetate), estrogens (diethylstilbestrol and ethinyl estradiol), antiestrogens (tamoxifen), and androgens (testosterone propionate and fluoxymesterone).

Drugs that interfere with intracellular protein synthesis can also be coupled to the ligand, such drugs are known to these skilled in the art and include puromycin, cycloheximide, and ribonuclease.

Toxins can also be coupled to the ligand. Toxins useful as therapeutics are known to those skilled in the art and include plant and bacterial toxins, such as, abrin, alpha toxin, diphtheria toxin, exotoxin, gelonin, pokeweed antiviral protein, ricin, and saporin.

Other therapeutic active agents include anti-DNA, anti-RNA, radiolabeled oligonucleotides, such as anti-sense oligodeoxyribonucleotides, anti-protein and anti-chromatin cytotoxic and or antimicrobial agents.

Suitable radioisotopes for coupling with the ligand to produce diagnostic or therapeutic active agents and used in diagnostic or therapeutic methods include, but are not limited to, actinium-225, astatine-211, iodine-120, iodine-123, iodine-124, iodine-125, iodine-126, iodine-131, iodine-133, bismuth-212, arsenic-72, bromine-75, bromine-76, bromine-77, indium-110, indium-111, indium-113m, gallium-67, gallium-68, strontium-83, zirconium-89, ruthenium-95, ruthenium-97, ruthenium-103, ruthenium-105, mercury-107, mercury-203, rhenium-186, rhenium-188, tellurium-121m, tellurium-122m, tellurium-125m, thulium-165, thulium-167, thulium-168, technetium-94m, technetium-99m, fluorine-18, silver-111, platinum-197, palladium-109, copper-62, copper-64, copper-67, phosphorus-32, phosphorus-33, yttrium-86, yttrium-90, scandium-47, samarium-153, lutetium-177, rhodium-105, praseodymium-142, praseodymium-143, terbium-161, holmium-166, gold-199, cobalt-57, cobalt-58, chromium-51, iron-59, selenium-75, thallium-201, and ytterbium-169, either individually or in combination thereof. Particularly, the radioisotope will emit a particle or ray in the 10-7,000 keV range, more particularly 50-1,500 keV.

Particular examples of therapeutic active agents include, but are not limited to, actinium-225, bismuth-212, lead-212, bismuth-213, iodine-125, iodine-131, rhenium-186, rhenium-188, silver-111, platinum-197, palladium-109, copper-67, copper-64, phosphorus-32, phosphorus-33, yttrium-90, scandium-47, samarium-153, lutetium-177, rhodium-105, praseodymium-142, praseodymium-143, terbium-161, holmium-166, and gold-199, either individually or in combination thereof.

Diagnostic Active Agents

Particular examples of diagnostic active agents for imaging applications include, but are not limited to, iodine-123, iodine-125, iodine-131, indium-111, gallium-67, ruthenium-97, technetium-99m, cobalt-57, cobalt-58, chromium-51, iron-59, selenium-75, thallium-201, ytterbium-169, copper-64, and fluorine-18, either individually or in combination thereof.

In one embodiment, the active agent labeled species further comprises a linker. The linker includes any linking moiety that attaches the ligand to the active agent through a first moiety. The linker can be as short as one carbon or a long polymeric species such as polyethylene glycol, polylysine or other polymeric species normally used in the pharmaceutical industry for modulating pharmacokinetic and biodistribution characteristics of active agents. Other linkers of varying length include C₁-C₂₅₀ length with one or more heteroatoms selected from O, S, N, P, and optionally substituted with halogen atoms. In a particular embodiment, the linker comprises at least one of an oligomeric or polymeric species made of natural or synthetic monomers, oligomeric or polymeric moiety selected from a pharmacologically acceptable oligomer or polymer composition, an oligo- or poly-amino acid, peptide, saccharide, a nucleotide, and an organic moiety with 1-250 carbon atoms, either individually or in combination thereof. The organic moiety with 1-250 carbon may contain one or more heteroatoms such as O, S, N or P and optionally substituted with halogen atoms at one or more places.

The first moiety may simply be an extension of the linker, formed by the reaction of a reactive species on the linker with a reactive group on the active agent, or a chelator that complexes the active agent. Examples of reactive species and the reactive group include, but are not limited to, activated esters (such as N-hydroxysuccinimide ester, pentafluorophenyl ester), a phosphoramidite, an isocyanate, an isothiocyanate, an aldehyde, an acid chloride, a sulfonyl chloride, a maleimide an alkyl halide, an amine, a phosphine, a phosphate, an alcohol or a thiol with the proviso that the reactive species and reactive group are matched to undergo a reaction yielding covalently linked conjugates. In a particular embodiment, the first moiety comprises a chelating moiety that is conjugated to the oligomeric or polymeric linker species, and the diagnostic active agent is capable of generating a signal that is detectable.

In one aspect, the diagnostic active agent is a magnetic resonance imaging contrast agent, which enhances the contrast of images obtained in magnetic resonance imaging procedure. Suitable paramagnetic ions that are useful for magnetic resonance imaging (“MRI”) are those of elements having atomic numbers of 21-29, 42, 44, and 58-70. Particularly useful are gadolinium ion and iron metal, ion, or oxides. Particularly, gadolinium ions are bound by chelators, such as polycarboxylic acids (carboxylic acids having a plurality of —COOH groups), which are conjugated directly or indirectly to the ligand through one of the —CO(O)— groups. Non-limiting examples of such chelators include diethylenetriamine-pentaacetic acid (“DTPA”); 1,4,7,10-tetraazacyclododecane-N,N′,N″,N′″-tetraacetic acid (“DOTA”); p-isothiocyanatobenzyl-1,4,7,10-tetraazacyclododecane-1,4,7,10-tetraacetic acid (“p-SCN-Bz-DOTA”); 1,4,7,10- tetraazacyclododecane-N,N′,N″-triacetic acid (“DO3A”); 1,4,7,10-tetraazacyclododecane-1,4,7,10-tetrakis(2-propionic acid) (“DOTMA”); 3,6,9-triaza-12-oxa-3,6,9-tricarboxymethylene-10-carboxy-1 3-phenyl-tridecanoic acid (“B-19036”); 1,4,7-triazacyclononane-N,N′,N″-triacetic acid (“NOTA”); 1,4,8,11-tetraazacyclotetradecane-N,N′,N″,N′″-tetraacetic acid (“TETA”); triethylene tetraamine hexaacetic acid (“TTHA”); trans-1,2-diaminohexane tetraacetic acid (=37 CYDTA”); 1,4,7,10-tetraazacyclododecane-1-(2-hydroxypropyl)4,7,10-triacetic acid (“HP-DO3A”); trans-cyclohexane-diamine tetraacetic acid (“CDTA”); trans(1,2)-cyclohexane diethylene triamine pentaacetic acid (“CDTPA”); 1-oxa-4,7,10-triazacyclododecane-N,N′,N″-triacetic acid (“OTTA”); 1,4,7,10-tetraazacyclododecane-1,4,7,10-tetrakis{3-(4-carboxyl)-butanoic acid}; 1,4,7,10-tetraazacyclododecane-1,4,7,10-tetrakis(acetic acid-methyl amide); 1,4,7,10-tetraazacyclododecane-1,4,7,10-tetrakis(methylene phosphonic acid); and derivatives thereof.

Superparamagnetic metals or oxides thereof, such as iron, chromium, cobalt, manganese, nickel, and tungsten oxide, are also suitable for generating magnetic resonance signal useful for diagnostic purposes, as disclosed, for example, in U.S. Pat. Nos. 5,492,814 and 5,314,679. Such metal oxides are particularly present in nanometer-sized aggregates (e.g. from about 10 nm to about 500 nm), either uncoated or coated, particularly with a shell comprising a biologically compatible material, such as polysaccharide, poly(amino acid), or organosilane. The coated or uncoated metal oxide aggregates are then covalently attached directly or indirectly (through a linker) to the ligand to produce a signal generating species. Methods for attachment of metal oxide (such as iron oxide) particles to organic materials, such as proteins, are known and disclosed for example, in U.S. Pat. Nos. 5,492,814 and 4,628,037.

Pretargeting Conjugate

Embodiments of the pretargeting conjugate are shown in FIG. 1 and FIG. 2 and represented by the schema below:

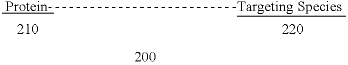

The protein is substantially free of a cofactor. In one embodiment, “substantially free of a cofactor” includes proteins that do not require any additional cofactor, chemical, chemical modification, or physical modification to be naturally stable under physiological conditions and room temperature and pressure in solution or as a solid, and can bind its corresponding ligand in vivo. Example of proteins include, but are not limited to, enzymes, soluble and serum proteins, proteins expressed on a surface of a cell, non-immunoglobulin proteins, intracellular proteins, and segment of proteins that are or can be made water-soluble, either individually or in combinations thereof as well as any derivatives of the proteins. Furthermore, when the pretargeting conjugate includes more than one protein, the proteins at each occurrence are independent of the proteins at every other occurrence.

In a particular embodiment, the protein includes such as, but not limited to, cysteine proteases, glutathione S transferase, epoxide hydrolase (EH), thiolase, NAD/NADP-dependent oxidoreductase, enoyl coA hydratase, aldehyde dehydrogenase, hydroxypyruvate reductase, tissue transglutaminase (tTG), formiminotransferase cyclodeaminase (FTCD), aminolevulinate A-dehydratase (ADD), creatin kinase, carboxylesterase (LCE), monoacylglycerol (MAG) lipase, metalloproteases (MP), phosphotases (protein tyrosine phosphotases, PTP), proteosome, FK506 binding protein (FKBP12), mammalian target of Rapamycin (mTOR; alternatively known as FKBP-rapamycin binding domain (FRB)), serine hydrolase (superfamily), ubiquitin-binding protein, β-galactosidase, nucleotide binding enzymes, protein kinases, GTP-binding proteins, cutinase, adenylosuccinate synthase, adenylosuccinate lyase, glutamate dehydrogenase, dihydrofolate reductase, fatty acid synthase, aspartate transcarbamylase, acetylcholinesterase, HMG cholate reductase, and cyclo-oxygenase (COX-1 and COX-2), either individually or in combinations thereof. Also included are any derivatives of any of the proteins.

In another example, the protein is covalently attached to the ligand.

Targeting Species

The targeting species 220 that is conjugated to the protein to form the pretargeting conjugate can be a compound or a fragment of a compound. As shown in FIG. 2, the targeting species 220 has one ore more a targeting moieties 222 that binds to a target sites 300 or to a substance produced by or associated with the target site via a primary binding site. Furthermore, as shown in FIG. 2, the targeting species may bind to one or more in-vivo targets 300 or a biomarker produced by or associated with the in-vivo-target. The target site is a specific site to which the active agent is to be delivered, such as a cell or group of cells, tissue, organ, tumor, or lesion. The targeting moiety binds to the target site or to a substance produced by or associated with the target site via a primary binding site. Non-limiting examples of targeting species include proteins, peptides, polypeptides, glycoproteins, lipoproteins, phospholipids, oligonucleotides, steroids, alkaloids or the like, e.g., hormones, lymphokines, growth factors, albumin, cytokines, enzymes, immune modulators, receptor proteins, oligonucleotides or mimics thereof, and antibodies and antibody fragments, either individually or in any combination thereof as well as derivatives thereof. Examples of particular targeting species include aptamers and thioaptamers. The targeting moieties preferentially bind marker substances that are produced by or associated with the target site.

Proteins are known that preferentially bind marker substances that are produced by or associated with lesions. For example, antibodies can be used against cancer-associated substances, as well as against any pathological lesion that shows an increased or unique antigenic marker, such as against substances associated with cardiovascular lesions, for example, vascular clots including thrombi and emboli, myocardial infarctions and other organ infarcts, and atherosclerotic plaques, inflammatory lesions, and infectious and parasitic agents.

Cancer states include carcinomas, melanomas, sarcomas, neuroblastomas, leukemias, lymphomas, gliomas, myelomas, and neural tumors.

Infectious diseases include those caused by invading microbes or parasites. As used herein, “microbe” denotes virus, bacteria, rickettsia, mycoplasma, protozoa, fungi and like microorganisms, “parasite” denotes infectious, generally microscopic or very small multicellular invertebrates, or ova or juvenile forms thereof, which are susceptible to antibody-induced clearance or lytic or phagocytic destruction, e.g., malarial parasites, spirochetes and the like, including helminths, while “infectious agent” or “pathogen” denotes both microbes and parasites.

The protein substances useful as targeting species include, but are not limited to, peptide, polypeptide, glycoprotein, lipoprotein, hormones, lymphokines, growth factors, albumin, cytokines, enzymes, immune modulators, receptor proteins, antibodies and antibody fragments, soluble and serum proteins, proteins expressed on a surface of a cell, segment of proteins that are or can be made water-soluble, non-immunoglobulin proteins, intracellular proteins, and derivatives thereof.

The protein substances of particular interest are antibodies and antibody fragments. The terms “antibodies” and “antibody fragments” mean generally immunoglobulins or fragments thereof that specifically bind to antigens to form immune complexes.

The antibody may be a whole immunoglobulin of any class; e.g., IgG, IgM, IgA, IgD, IgE, chimeric or hybrid antibodies with dual or multiple antigen or epitope specificities. It can be a polyclonal antibody, particularly a humanized or affinity-purified antibody from a human. It can be an antibody from an appropriate animal; e.g., a primate, goat, rabbit, mouse, or the like. If the target site-binding region is obtained from a non-human species, the target species may be humanized to reduce immunogenicity of the non-human antibodies, for use in human diagnostic or therapeutic applications. Such a humanized antibody or fragment thereof is also termed “chimeric.” For example, a chimeric antibody comprises non-human (such as murine) variable regions and human constant regions. A chimeric antibody fragment can comprise a variable binding sequence or complementarity-determining regions (“CDR”) derived from a non-human antibody within a human variable region framework domain. Monoclonal antibodies are also suitable because of their high specificities. Monoclonal antibodies are readily prepared by what are now considered conventional procedures of immunization of mammals with an immunogenic antigen preparation, fusion of immune lymph or spleen cells with an immortal myeloma cell line, and isolation of specific hybridoma clones. More unconventional methods of preparing monoclonal antibodies are not excluded, such as interspecies fusions and genetic engineering manipulations of hypervariable regions, since it is primarily the antigen specificity of the antibodies that affects their utility in the present invention. It will be appreciated that newer techniques for production of monoclonal antibodies (“MAb”) can also be used; e.g., human MAbs, interspecies MAbs, chimeric (e.g., human/mouse) MAbs, genetically engineered antibodies, and the like.

Useful antibody fragments include F(ab′)₂, F(ab)₂, Fab′, Fab, Fv, and the like including hybrid fragments. Particular fragments are Fab′, F(ab′)₂, Fab, and F(ab)₂. Also useful are any subfragments retaining the hypervariable, antigen-binding region of an immunoglobulin and having a size similar to or smaller than a Fab′ fragment. An antibody fragment can include genetically engineered and/or recombinant proteins, whether single-chain or multiple-chain, which incorporate an antigen-binding site and otherwise function in-vivo as targeting species in substantially the same way as natural immunoglobulin fragments. Such single-chain binding molecules are disclosed in U.S. Pat. No. 4,946,778. Fab′ antibody fragments may be conveniently made by reductive cleavage of F(ab′)₂ fragments, which themselves may be made by pepsin digestion of intact immunoglobulin. Fab antibody fragments may be made by papain digestion of intact immunoglobulin, under reducing conditions, or by cleavage of F(ab)₂ fragments which result from careful papain digestion of whole immunoglobulin. The fragments may also be produced by genetic engineering.

It should be noted that mixtures of antibodies and immunoglobulin classes can be used, as can hybrid antibodies. Multispecific, including bispecific and hybrid, antibodies and antibody fragments are sometimes desirable in the present invention for detecting and treating lesions and comprise at least two different substantially monospecific antibodies or antibody fragments, wherein at least two of said antibodies or antibody fragments specifically bind to at least two different antigens produced or associated with the targeted lesion or at least two different epitopes or molecules of a marker substance produced or associated with the targeted lesion. Multispecific antibodies and antibody fragments with dual specificities can be prepared analogously to the anti-tumor marker hybrids disclosed in U.S. Pat. No. 4,361,544. Other techniques for preparing hybrid antibodies are disclosed in; e.g., U.S. Pat. Nos. 4,474,893 and 4,479,895, and in Milstein et al., Immunology Today, Vol. 5, 299 (1984).

Particular proteins that may be used are proteins having a specific immunoreactivity to a biomarker substance of at least 60% and a cross-reactivity to other antigens or non-targeted substances of less than 35%.

As disclosed above, antibodies against tumor antigens and against pathogens are known. For example, antibodies and antibody fragments which specifically bind biomarkers produced by or associated with tumors or infectious lesions, including viral, bacterial, fungal and parasitic infections, and antigens and products associated with such microorganisms have been disclosed, inter alia, in Hansen et al. (U.S. Pat. No. 3,927,193) and Goldenberg (U.S. Pat. Nos. 4,331,647, 4,348,376, 4,361,544, 4,468,457, 4,444,744, 4,818,709 and 4,624,846). In particular, antibodies against an antigen, e.g., a gastrointestinal, lung, breast, prostate, ovarian, testicular, brain or lymphatic tumor, a sarcoma, or a melanoma, are advantageously used.

A wide variety of monoclonal antibodies against infectious disease agents have been developed, and are summarized in a review by Polin, in Eur. J. Clin. Microbiol., 3(5):387-398, 1984, showing ready availability. These include MAbs against pathogens and their antigens. Exemplary infectious disease agents are disclosed in U.S. Pat. No. 5,482,698.

Additional examples of MAbs generated against infectious organisms that have been described in the literature are noted below.

MAbs against the gp 120 glycoprotein antigen of human immunodeficiency virus 1 (HIV-1) are known, and certain of such antibodies can have an immunoprotective role in humans. See, e.g., Rossi et al., Proc. Natl. Acad. Sci. USA, Vol. 86, pp. 8055-58 (1990). Other MAbs against viral antigens and viral-induced antigens are also known. MAbs against malaria parasites can be directed against the sporozoite, merozoite, schizont and gametocyte stages.

Suitable MAbs have been developed against most of the microorganisms (bacteria, viruses, protozoa, other parasites) responsible for the majority of infections in humans, and many have been used previously for in vitro diagnostic purposes. These antibodies, and newer MAbs that can be generated by conventional methods, are appropriate for use in the present invention.

Proteins useful for detecting and/or treating cardiovascular lesions include fibrin-specific proteins; for example, fibrinogen, soluble fibrin, antifibrin antibodies and fragments, fragment E₁ (a 60 kDa fragment of human fibrin made by controlled plasmin digestion of crosslinked fibrin), plasmin (an enzyme in the blood responsible for the dissolution of fresh thrombi), plasminogen activators (e.g., urokinase, streptokinase and tissue plasminogen activator), heparin, and fibronectin (an adhesive plasma glycoprotein of 450 kDa) and platelet-directed proteins; for example, platelets, antiplatelet antibodies, and antibody fragments, anti-activated platelet antibodies, and anti-activated platelet factors, which have been reviewed by Koblik et al., Semin. Nucl. Med., Vol. 19, 221-237 (1989).

In one embodiment, the targeting species is an MAb or a fragment thereof that recognizes and binds to a heptapeptide of the amino terminus of the β-chain of fibrin monomer. Fibrin monomers are produced when thrombin cleaves two pairs of small peptides from fibrinogen. Fibrin monomers spontaneously aggregate into an insoluble gel, which is further stabilized to produce blood clots.

In another embodiment, the targeting species is a chimeric antibody derived from an antibody designated as NR-LU-10. This chimeric antibody has been designated as NR-LU-13 and disclosed in U.S. Pat. No. 6,358,710. NR-LU-13 contains the murine Fv region of NR-LU-10 and therefore comprises the same binding specificity as NR-LU-10. It also comprises human constant regions. Thus, this chimeric antibody binds the NR-LU-10 antigen and is less immunogenic because it is made more human-like. NR-LU-10 is a nominal 150 kilodalton (or kDa) murine IgG_{2b} pan carcinoma monoclonal antibody that recognizes an approximately 40 kDa glycoprotein antigen expressed on most carcinomas, such as small cell lung, non-small cell lung, colon, breast, renal, ovarian, pancreatic, and other carcinoma tissues. The NR-LU-10 antigen has been further described by Varki et al., “Antigens Associated With a Human Lung Adenocarcinoma Defined by Monoclonal Antibodies,” Cancer Research, Vol. 44, 681-87 (1984), and Okabe et al.,” “Monoclonal Antibodies to Surface Antigens of Small Cell carcinoma of the Lungs,” Cancer Research Vol. 44, 5273-78 (1984). Methods for preparing antibodies that binds to epitopes of the NR-LU-10 antigen are known and are disclosed in U.S. Pat. No. 5,084,396. One suitable method for producing monoclonal antibodies is the standard hybridoma production and screening process, which is well known in the art. In a particular embodiment, the targeting species is a humanized antibody or humanized antibody fragment that binds specifically to the antigen bound by antibody NR-LU-13. A humanization method comprises grafting only non-human CDRs onto human framework and constant regions (see; e.g., Jones et al., Nature, Volume 321, 522-35 (1986)). Another humanization method comprises transplanting the entire non-human variable domains, but cloaking (or veneering) these domains by replacement of exposed residues reduce immunogenicity (see; e.g., Padlan, Molec. Immun., Vol. 28, 489-98 (1991)). Exemplary humanized light and heavy sequences derived from the light and heavy sequences of the NR-LU-13 antibody are disclosed in U.S. Pat. No. 6,358,710, and are denoted therein as NRX451. The phrase “binds specifically” with respect to antibody or antibody fragment means such antibody or antibody fragment has a binding affinity of at least about 10⁴ M⁻¹. Particularly, the binding affinity is at least about 10⁶ M⁻¹, and more particularly, at least about 10⁸ M⁻¹.

According to still another embodiment, the targeting species is a humanized anti-p185^{HER2} antibody that specifically recognizes the pl85 HER2 protein expressed on breast cancer cells. A humanized anti-p185^{HER2} antibody known as Herceptin is widely available. An anti-HER2 murine MAb known as ID5 is available from Applied BioTechnology/Oncogene Science (Cambridge, Mass.), which can be humanized according to conventional methods. See, e.g., X. F. Lee et al., “Differential Signaling by an Anti-p185^{HER2} Antibody and Hergulin,” Cancer Research, Vol. 60, 3522-31 (2000).

In other embodiments, the targeting species is an antibody or a fragment thereof, particularly a humanized antibody or fragment thereof, that is raised against one of anti-carcinogembryonic antigen (“CEA”), anti-colon-specific antigen-p (“CSAp”), and other well known tumor-associated antigens, such as CD19, CD 20, CD21, CD22, CD23, CD30, CD74, CD80, HLA-DR, I, MUC 1, MUC 2, MUC 3, MUC 4, EGFR, HER2/neu, PAM-4, Bre3, TAG-72 (C72.3, CC49), EGP-1 (e.g., RS7), EGP-2 (e.g., 17-1A and other Ep-CAM targets), Le(y (e.g., B3), A3, KS-1, S100, IL-2, T101, necrosis antigens, folate receptors, angiogenesis markers (e.g., VEGFR), tenascin, PSMA, PSA, tumor-associated cytokines, MAGE and/or fragments thereof. Tissue-specific antibodies (e.g., against bone marrow cells, such as CD34, CD74, etc., parathyroglobulin antibodies, etc.) as well as antibodies against non-malignant diseased biomarkers, such as macrophage antigens of atherosclerotic plaques (e.g., CD74 antibodies), and also specific pathogen antibodies (e.g., against bacteria, viruses, and parasites) are well known in the art.

It should be understood that the foregoing disclosure of various antigens or biomarkers that can be used to raise specific antibodies against them (and from which antibodies fragments may be prepared) serves only as examples, and is not to be construed in any way as a limitation of the present invention.

The compounds of the present invention, for example, the active agent-labeled species, the pretargeting conjugate, or both, can be incorporated into pharmaceutical compositions suitable for administration into a subject, which pharmaceutical compositions comprise a pharmaceutically acceptable carrier. As used herein, “pharmaceutically acceptable carrier” includes any and all solvents, dispersion media, coatings, antibacterial and antifungal agents, isotonic and absorption delaying agents, and the like that are physiologically compatible with the subject. Particularly, the carrier is suitable for intravenous, intramuscular, subcutaneous, or parenteral administration (e.g., by injection). Depending on the route of administration, the active agent-labeled species, the pretargeting conjugate, or both, may be coated in a material to protect the compound or compounds from the action of acids and other natural conditions that may inactivate the compound or compounds.

In yet another embodiment, the pharmaceutical composition comprising the active agent-labeled species, the pretargeting conjugate, or both, and a pharmaceutically acceptable carrier can be administered by combination therapy, i.e., combined with other agents. For example, the combination therapy can include a composition of the present invention with at least a therapeutic agent or drug, such as an anti-cancer or an antibiotic. Exemplary anti-cancer agents include cis-platin, adriamycin, and taxol. Exemplary antibiotics include isoniazid, rifamycin, and tetracycline.

Methods for Diagnosing or Treating Diseases Using Pretargeting Strategy

With reference to FIG. 3, next will be described a method for diagnosing, detecting, and/or treating a disease condition by preferentially delivering a diagnostic active agent to the site of the disease. FIG. 3 is a flow chart of the method. The method includes, at Step 305, administering a pretargeting conjugate to a subject. At Step 315, the pretargeting conjugate is allowed to localize at the target. Step 325 includes administering an active agent-labeled species to the subject. The active agent-labeled species includes a ligand coupled with an active agent selected from a group consisting of diagnostic active agents, therapeutic active agents, and combinations thereof. The protein is substantially free of a cofactor. The active agent is capable of performing a function selected from elucidating the disease condition and reducing an adverse effect of the disease condition.

In one embodiment, the targeting species is an antibody or a fragment thereof that binds to an antigen present at the target, which can be a diseased cell or tissue, or a marker substance produced by the diseased cell or tissue. The active agent is a moiety that generate a unique signal that is recognizable by diagnostic medical imaging techniques, such as MRI, PET, SPECT, X-ray imaging, CT, ultrasound imaging, or optical imaging.

In another embodiment, the active agent is a radioisotope, drug, toxin, fluorescent dye activated by nonionizing radiation, hormone, hormone antagonist, receptor antagonist, enzyme or proenzyme activated by another agent, autocrine, or cytokine.

In one aspect, the active agent-labeled species comprises a chelator (e.g. DTPA) which forms a coordination complex with paramagnetic ions, (e.g., Gd³⁺) for MRI application. The active agent-labeled species may be administered into the patient at a dose from about 0.01 to about 0.05 moles Gd/kg of body weight of the patient. An MRI system that can be used for practicing a method of the present invention is disclosed in U.S. Pat. No. 6,235,264. The pair of pharmaceuticals of the present invention is formulated with a physiologically acceptable carrier, such as an intravenous fluid, for intravenously administering into the patient. These pharmaceuticals may also be administered orally under appropriate circumstances.

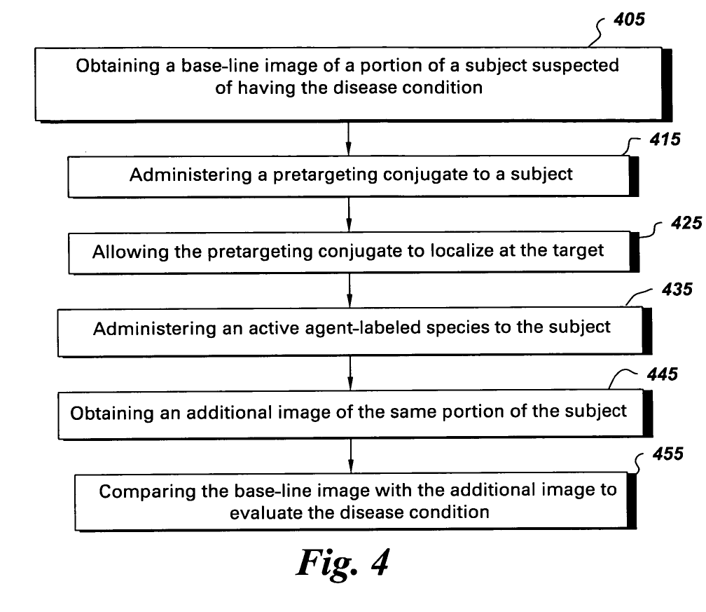

With reference to FIG. 4, next will be described another method for diagnosing, detecting, and/or treating a disease condition. The method includes, at Step 405, obtaining one or more base-line image of a portion of a subject suspected of having the disease condition. Image as used herein includes signals such as, but not limited to, visual representation of the spatial distribution (or location) of an object. In one embodiment, the image consists of an array of more than one dimension, where the values of the array typically represent an intensity associated with a spatial coordinate in two or three dimensions. Step 415 includes administering a pretargeting conjugate to the subject. Step 425 includes allowing the pretargeting conjugate to localize at the target. Step 435 includes administering an active agent-labeled species to the subject. Step 445 includes obtaining one or more additional image of the same portion of the subject. Step 455 includes comparing the base-line image with the additional image to evaluate the disease condition. The pretargeting conjugate includes a protein conjugated to a targeting species having a targeting moiety that binds to an in-vivo target or a marker substance produced by or associated with the target. The active agent-labeled species includes a ligand coupled with an active agent selected from a group consisting of diagnostic active agents, therapeutic active agents, and combinations thereof. The protein is substantially free of a cofactor. The active agent is capable of performing a function selected from a group consisting of elucidating the disease condition and reducing an adverse effect of the disease condition.

The step of obtaining additional images to evaluate the disease condition may be repeated at different time intervals as desired. Thus, it should be appreciated that one or more base line images may be compared with one or more additional images or the additional images may be compared with each other.

Another aspect of the invention provides a method for assessing an effectiveness of a prescribed regimen for treating a disease that is characterized by an overproduction or underproduction of a disease-specific substance or biomarker. The method includes: (i) obtaining a base-line image of a portion of a subject suspected to carry the disease; (ii) administering a pretargeting conjugate to the subject; (iii) allowing the pretargeting conjugate to localize at the target; and (iv) administering an active agent-labeled species to the subject; (v) obtaining a pre-treatment image coming from the same portion of the subject; (vi) treating the disease condition in the subject with a prescribed regimen; (vii) repeating steps (ii), (iii), and (iv); and (viii) obtaining a post-treatment image coming from the same portion of the subject as in step (v).

The method may further comprise comparing the post-treatment image to the pre-treatment image to assess the effectiveness of the prescribed regimen, wherein a change in image contrast during a course of the prescribed regimen indicates that the treatment has provided benefit. The method may also further comprise comparing the post-treatment image to the baseline image to assess the effectiveness of the prescribed regimen, wherein a change in image contrast or signals during a course of the prescribed regimen indicates that the treatment has provided benefit. The method may also further comprising repeating steps (vii) and (viii) at predetermined time intervals during the course of treating the disease condition.

In various aspects of the methods, any one of the pretargeting conjugates and active agent-labeled species that are specifically described above can be chosen to suit the particular circumstances and disease.

During the course of the treatment of the disease, a change in signal obtained from the imaging technique (compared to a base-line signal obtained before the treatment) of, for example, 10 percent or more can signify that the treatment has conferred some benefit. In another embodiment, a change in signal obtained from the imaging technique (compared to a base-line signal obtained before the treatment) of, for example, 20 percent or more can signify that the treatment has conferred some benefit. The prescribed regimen for treating the disease can be, for example, treatment with drugs, radiation, or surgery.

In one aspect, the present invention provides a kit that comprises the active agent-labeled species and the pretargeting conjugate kept separately before use for purposes of diagnosing or treating diseases.

### EXAMPLES

### Example 1

Preparation Of Pretargeting Conjugate

A monoclonal antibody (i.e. targeting species) against a biomarker of cancer, (such as Her2, urokinase receptor, carcinoembryonic antigen or other) could be expressed in hybridoma cell lines and purified from culture using affinity chromatography methods. For example, antibodies could be purified by capture on Protein G columns and eluted from column by addition of 100 mM glycine buffer at pH 3.0. The pure antibody could then be chemically coupled to a purified protein/ enzyme (such as a serine esterase) using known methods to those skilled in the art of bioconjugation (NHS ester, disulfide bond formation, amide bond formation). The resulting pretargeting conjugate containing the targeting antibody and the capturing enzyme would be further purified from the unconjugated components using conventional chromatography techniques.

In another example, a genetic construct could be designed so that the targeting species is coded upstream or downstream of the protein/ enzyme to express the complete pretargeting conjugate in cells. The genetic construct would include promoter elements that would allow for overexpression in cells, such as bacteria, yeast, or mammalian. The constructs could also code for affinity tags (Histidine tags, GST fusion, or others) upstream or downstream of the pretargeting conjugate that would allow for rapid purification from cell lysate following overexpression of the pretargeting conjugate. The pretargeting conjugate would be purified to homogeneity by methods known to those familiar in the art of protein purification.

### Example 2

Labeling the Active Agent Species

In one example of the active agent labeled species, the ligand is an alkyl phosphonate and the active agent (i.e. label) is 18F. The carbon chain from the central phosphorous atom contains several sites for fluorination by methods known to those skilled in the art. The source of fluorine would be 18F and allow for 18F fluorination of the ligand, which could then be purified from reaction mixture or administered as a solution.

### Example 3

Pretargeting Conjugate Interaction With Active Agent-Labeled Species

Methods exist to determine the interaction of the active agent labeled species with the pretargeting conjugate. In one example, cells expressing the biomarker of interest could be cultured to use in a binding assay. The cells could derive from human tumors (ATCC cell nuber LS 174T), which overexpress carcinoembryonic antigen. The purified pretargeting conjugate could be added to the cells in solution at a concentration of 1 milligram per mL and incubated in culture conditions for an hour or more. The unbound portion of the pretargeting conjugate would then be washed away with sterile buffer and culture media would be replaced. The active agent labeled species could then be added to cells that have and have not been incubated with the pretargeting conjugate and incubated for an additional optimal period of time. The unbound active agent labeled species would then be washed away with sterile buffer. The results of binding between the active agent labeled species and the pretargeting conjugate could be confirmed by counting of the radioactive signal from 18F by a gamma counter or by autoradiography.

### Example 4

Method of Administration

Purified pretargeting conjugate would be suspended in a sterile solution of aqueous buffer in preparation for intravenous injection into a subject. The pretargeting conjugate would then be injected at an optimal dose in an optimal volume to have little to no adverse effect in subjects. After a predetermined amount of time for clearance of unbound pretargeting construct, the active agent species would be fluorinated or otherwise labeled for imaging and injected intravenously to the subject. The subject would then be monitored for adverse effects until an optimal time has passed for effective conjugation of the active agent labeled species to the pretargeting conjugate. The subject will then be imaged to determine the efficacy of the pretargeting conjugate:active agent-labeled species combination.

While the invention has been described in detail in connection with only a limited number of aspects, it should be readily understood that the invention is not limited to such disclosed aspects. Rather, the invention can be modified to incorporate any number of variations, alterations, substitutions or equivalent arrangements not heretofore described, but which are commensurate with the spirit and scope of the invention. Additionally, while various embodiments of the invention have been described, it is to be understood that aspects of the invention may include only some of the described embodiments. Accordingly, the invention is not to be seen as limited by the foregoing description, but is only limited by the scope of the appended claims.

What is claimed is:

1. A set of compounds comprising an active agent-labeled species and a pretargeting conjugate,
wherein the active agent-labeled species comprises a ligand coupled with an active agent selected from a group consisting of diagnostic active agents, therapeutic active agents, and combinations thereof;
wherein the pretargeting conjugate comprises a protein that is conjugated to a targeting species having a targeting moiety capable of binding to an in-vivo target or a biomarker substance produced by or associated with the target; and
wherein the protein is substantially free of a cofactor.

2. The set of compounds of claim 1, wherein the ligand comprises at least one ligand selected from a group consisting of a sulfonate ester derivative, α-chloroacetamide derivative, an α-chloroacetamide derivative on a peptide, a fluorophosphonate or a fluorophosphate derivative, a matrix metalloprotease inhibitor ilomastat, zinc-chelating hydroxamate coupled to benzophenone photocrosslinker, sublactam, DFPP, α-bromobenzylphosphonate, β-lactamase inhibitor, rapamycin, FK506 FK1012, AP1510, AP1903, AP20187, ubiquitin, tyr-phosphate mimic, 5′-p-fluorosulfonylbenzoyl adenosine, fatty acid synthase inhibitor, and combinations thereof.

3. The set of compounds of claim 1, wherein the active-agent comprises an isotope selected from a group consisting of actinium-225, astatine-211, iodine-120, iodine-123, iodine-124, iodine-125, iodine-126, iodine-131, iodine-133, bismuth-212, arsenic-72, bromine-75, bromine-76, bromine-77, indium-110, indium-11, indium-113m, gallium-67, gallium-68, strontium-83, zirconium-89, ruthenium-95, ruthenium-97, ruthenium-103, ruthenium-105, mercury-107, mercury-203, rhenium-186, rhenium-188, tellurium-121m, tellurium-122m, tellurium-125m, thulium-165, thulium-167, thulium-168, technetium-94m, technetium-99m, fluorine-18, silver-111, platinum-197, palladium-109, copper-62, copper-64, copper-67, phosphorus-32, phosphorus-33, yttrium-86, yttrium-90, scandium-47, samarium-153, lutetium-177, rhodium-105, praseodymium-142, praseodymium-143, terbium-161, holmium-166, gold-199, cobalt-57, cobalt-58, chromium-51, iron-59, selenium-75, thallium-201, ytterbium-169, and combinations thereof.

4. The set of compounds of claim 1, wherein the active agent labeled species further comprises a linker having a first moiety, wherein the ligand is associated with the linker.

5. The set of compounds of claim 4, wherein the linker is coupled to an active agent that generates a detectable signal.

6. The set of compounds of claim 4, wherein the first moiety comprises a chelating moiety.

7. The set of compounds of claim 6, wherein the chelating moiety is selected from a group consisting of diethylenetriamine-pentaacetic acid (“DTPA”), 1,4,7,10-tetraazacyclododecane-N,N′,N″,N′″-tetraacetic acid (“DOTA”), p-isothiocyanatobenzyl-1,4,7,10-tetraazacyclododecane-1,4,7,10-tetraacetic acid (“p-SCN-Bz-DOTA”), 1,4,7,10- tetraazacyclododecane-N,N′,N″-triacetic acid (“DO3A”), 1,4,7,10-tetraazacyclododecane-1,4,7,10-tetrakis(2-propionic acid) (“DOTMA”), 3,6,9-triaza-12-oxa-3,6,9-tricarboxymethylene-10-carboxy-13-phenyl-tridecanoic acid (“B-19036”), 1,4,7-triazacyclononane-N,N′,N″-triacetic acid (“NOTA”), 1,4,8,11-tetraazacyclotetradecane-N,N′,N″,N′″-tetraacetic acid (“TETA”), triethylene tetraamine hexaacetic acid (“TTHA”), trans-1,2-diaminohexane tetraacetic acid (“CYDTA”), 1,4,7,10-tetraazacyclododecane-1-(2-hydroxypropyl)4,7,10-triacetic acid (“HP-DO3A”), trans-cyclohexane-diamine tetraacetic acid (“CDTA”), trans(1,2)-cyclohexane diethylene triamine pentaacetic acid (“CDTPA”), 1-oxa-4,7,10-triazacyclododecane-N,N′,N″-triacetic acid (“OTTA”), 1,4,7,10-tetraazacyclododecane-1,4,7,10-tetrakis {3-(4-carboxyl)-butanoic acid }, 1,4,7,10-tetraazacyclododecane-1,4,7,10-tetrakis(acetic acid-methyl amide), 1,4,7,10-tetraazacyclododecane-1,4,7,10-tetrakis(methylene phosphonic acid), and derivatives thereof; and the chelating moiety forms a coordination complex with a paramagnetic species.

8. The set of compounds of claim 1, wherein the protein comprises at least one protein selected from a group consisting of enzymes, soluble and serum proteins, proteins expressed on a surface of a cell, segment of proteins that are or can be made water-soluble, non-immunoglobulin proteins, intracellular proteins, and derivatives thereof.

9. The set of compounds of claim 1, wherein the protein comprises at least one protein selected from a group consisting of cysteine proteases, glutathione S transferase, epoxide hydrolase (EH), thiolase, NAD/NADP-dependent oxidoreductase, enoyl coA hydratase, aldehyde dehydrogenase, hydroxypyruvate reductase, tissue transglutaminase (tTG), formiminotransferase cyclodeaminase (FICD), aminolevulinate D-dehydratase (ADD), creatin kinase, carboxylesterase (LCE), monoacylglycerol (MAG) lipase, metalloproteases (MP), phosphotases (protein tyrosine phosphotases, PTP), proteosome, FK506, FKRB, serine hydrolase (superfamily), ubiquitin-binding protein, β-galactosidase, nucleotide binding enzymes, protein kinases, GTP-binding proteins, cutinase, adenylosuccinate synthase, adenylosuccinate lyase, glutamate dehydrogenase, dihydrofolate reductase, fatty acid synthase, aspartate transcarbamylase, acetylcholinesterase, HMG cholate reductase, cyclo-oxygenase (COX-1 and COX-2), and combinations thereof.

10. The set of compounds of claim 1, wherein the active agent is paramagnetic Gd³⁺.

11. The set of compounds of claim 1, wherein the active agent is a therapeutic agent selected from a group consisting of isotopes, drugs, toxins, fluorescent dyes activated by nonionizing radiation, hormones, hormone antagonists, receptor antagonists, enzymes or proenzymes activated by another agent, autocrine, cytokines, and combinations thereof.

12. The set of compounds of claim 11, wherein the therapeutic agent is selected from a group consisting of taxol, nitrogen mustards, cyclophosphamide, melphalan, uracil mustard, chlorambucil, ethylenimine derivatives, alkyl sulfonates, nitrosoureas, triazenes, folic acid analogs, pyrimidine analogs, purine analogs, vinca alkaloids, antibiotics, enzymes, platinum coordination complexes, substituted urea, methyl hydrazine derivatives, adrenocortical suppressants, hormones, antagonists, and combinations thereof.

13. The set of compounds of claim 1, wherein the targeting species is selected from a group consisting of proteins, peptides, polypeptides, glycoproteins, lipoproteins, phospholipids, oligonucleotides, steroids, hormones, lymphokines, growth factors, albumin, cytokines, enzymes, immune modulators, receptor proteins, oligonucleotides or mimics thereof, antibodies, antibody fragments, and derivatives thereof.

14. The set of compounds of claim 13, wherein the targeting species is selected from a group consisting of antibodies and fragments thereof.

15. The set of compounds of claim 14, wherein the antibodies and fragments thereof is selected from a group consisting of human or humanized antibodies, human or humanized antibody fragments, and combinations thereof.

16. The set of compounds of claim 1, wherein the biomarker is associated with a target selected from a group consisting of tumors, cardiovascular lesions, vascular clots, thrombi, emboli, myocardial infarctions, atherosclerotic plaques, inflammatory lesions, infectious and parasitic agents, and combinations thereof.

17. A method for diagnosing or treating a disease condition comprising:
i) administering a pretargeting conjugate to a subject, wherein the pretargeting conjugate comprises a protein conjugated to a targeting species having a targeting moiety that binds to an in-vivo target or a biomarker substance produced by or associated with the in-vivo target, and
wherein the protein is substantially free of a cofactor;
ii) allowing the pretargeting conjugate to localize at the in-vivo target; and
iii) administering an active agent-labeled species to the subject, wherein the active agent-labeled species comprises an active agent coupled with a ligand; and
wherein the active agent is capable of performing a function selected from a group consisting of elucidating the disease condition and reducing an adverse effect of the disease condition.

18. The method of claim 17, wherein the active agent is capable of generating a signal that is detectable.

19. The method of claim 17, wherein the active agent is a therapeutic agent selected from a group consisting of radioisotopes, drugs, toxins, fluorescent dyes activated by nonionizing radiation, hormones, hormone antagonists, receptor antagonists, enzymes, proenzymes activated by another agent, authorizes, cytokines, and combinations thereof.

20. A method for diagnosing or treating a disease condition comprising:
i) obtaining a base-line image from a portion of a subject suspected to have the disease condition;
ii) administering a pretargeting conjugate to the subject, wherein the pretargeting conjugate comprises a protein conjugated to a targeting species having a targeting moiety that binds to an in-vivo target or a biomarker substance produced by or associated with the in-vivo target, and wherein the protein is substantially free of a cofactor;
iii) allowing the pretargeting conjugate to localize at the target; and
iv) administering an active agent-labeled species to the subject, wherein the active agent-labeled species comprises an active agent coupled with a ligand; and
wherein the active agent is capable of performing a function selected from a group consisting of elucidating the disease condition and reducing an adverse effect of the disease condition.
v) obtaining an additional image from the same portion of the subject; and
vi) comparing the base-line image with the additional image to evaluate the disease condition.

21. A method for assessing an effectiveness of a prescribed regimen for treating a disease condition that is characterized by an overproduction or underproduction of a disease-specific substance or biomarker, the method comprising:
i) obtaining at least a base-line image and a base-line signal from a portion of a subject suspected of having the disease condition;
ii) administering a pretargeting conjugate to the subject, wherein the pretargeting conjugate comprises a protein conjugated to a targeting species having a targeting moiety that binds to an in-vivo target or a marker substance produced by or associated with the target, and wherein the protein is substantially free of a cofactor; and
iii) allowing the pretargeting conjugate to localize at the target;
iv) administering an active agent-labeled species to the subject, wherein the active agent-labeled species comprises an active agent coupled with a ligand;
wherein the active agent is capable of performing a function selected from a group consisting of elucidating the disease condition and reducing an adverse effect of the disease condition;
v) obtaining a pre-treatment image coming from the same portion of the subject;
vi) treating the disease condition in the subject with a prescribed regimen;
vii) repeating steps (ii), (iii), and (iv); and
viii) obtaining a post-treatment image coming from the same portion of the subject as in step (v).

22. The method of claim 21, further comprising comparing the post-treatment image to the pre-treatment image to assess the effectiveness of the prescribed regimen, wherein a decrease in image contrast or signals during a course of the prescribed regimen indicates that the treatment has provided benefit.

23. The method of claim 21, further comprising comparing the post-treatment image to the base-line image to assess the effectiveness of the prescribed regimen, wherein a decrease in image contrast or signals during a course of the prescribed regimen indicates that the treatment has provided benefit.

24. The method of claim 21, wherein the active agent is capable of generating a signal that is detectable

25. The method of claim 21, further comprising repeating steps (vii) and (viii) at predetermined time intervals during the course of treating the disease condition.
